(19) United States
(12) Patent Application Publication
(10) Pub. No.: US 2024/0391861 A1
(43) Pub. Date: Nov. 28, 2024

(54) PROCESS FOR THE RECOVERY OF MONOPOTASSIUM GLUCARATE AND GLUCARIC ACID

(71) Applicant: Alliance for Sustainable Energy, LLC, Golden, CO (US)

(72) Inventors: Hoon CHOI, Lakewood, CO (US); Eric M. KARP, Lakewood, CO (US); Nathan Edward SOLAND, Bakersfield, CA (US)

(21) Appl. No.: 18/704,534

(22) PCT Filed: Oct. 28, 2022
(86) PCT No.: PCT/US22/48260

§ 371 Date: Apr. 25, 2024

Related U.S. Application Data
(60) Provisional application No. 63/273,234, filed on Oct. 29, 2021.

(51) Int. Cl. C07C 51/46; C07C 51/48

(52) U.S. Cl. CPC C07C 51/46; C07C 51/48

(57) ABSTRACT

The processes disclosed herein for separation of glucaric acid via antisolvent crystallization and azeotropic drying separate monopotassium glucarate and glucaric acid with a recovery yield of greater than 99.9% and 71% at purities of about 95.6% and 98.3%, respectively. Processes disclosed herein recycle antisolvents such as IPA and acetone with greater than 99% recovery with an energy consumption of about 20 MJ/kg for isolation of potassium glucarate and 1,456 MJ/kg for glucaric acid. Using methods and processes disclosed herein, other oxygenated bio-carboxylic acids (e.g., mevalonic acid) can be separated and recovered from fermentation broths and abiotic reaction solutions.

### CROSS-REFERENCE TO RELATED APPLICATIONS

This application is a national phase entry under 35 U.S.C. § 371 and claims priority to PCT application number PCT/US2022/048260 filed 28 Oct. 2022 which claims priority under 35 U.S.C. § 119 to U.S. provisional patent application No. 63/273,234 filed on 29 Oct. 2021, the contents of which are hereby incorporated herein in their entirety.

### CONTRACTUAL ORIGIN

The United States Government has rights in this invention under Contract No. DE-AC36-08GO28308 between the United States Department of Energy and Alliance for Sustainable Energy, LLC, the Manager and Operator of the National Renewable Energy Laboratory.

### BACKGROUND

Glucaric acid and its salts have applications in many products such as detergents, corrosion inhibitors, and polymers. Its dicarboxylic acid functionality and 6-carbon chain make it a precursor to adipic acid, which is used for the production of nylons and biodegradable polymers such as polybutylene succinate adipate and polybutylene adipate terephthalate. It is also a promising additive in polymers such as polyvinyl alcohol, where 3-5 wt. % of glucaric acid has been shown to lower the melting temperature and improve mechanical performance. Furthermore, glucaric acid (GA) acts as a chelating agent for divalent ions (e.g. Ca²⁺ and Mg²⁺) such that GA can be used for phosphate-free and biodegradable detergents, or as a corrosion inhibitor in waste water treatment systems.

Today, GA is primarily produced via the chemical oxidation of glucose using nitric acid, an expensive and nonselective process, in which competing side reactions result in low isolated yields (≤43%) of GA. This highly exothermic oxidation requires a 4:1 molar ratio of nitric acid to glucose, which generates 0.85 kg of nitric acid waste per kg of GA and prevents commodity level production of GA through chemical catalysis. Alternatively, other GA production methods via electrochemical or catalytic oxidation methods with homogeneous or heterogenous catalysts have been studied, but these approaches were at small scale (<100 ml) and are actively being researched. In these reactions, organic acid byproducts such as gluconic acid, glucuronic acid, tartaric acid, and oxalic acid are often coproduced and result in a dilute and difficult solution to selectively isolate GA from.

### SUMMARY

In an aspect, disclosed herein is a method for isolating monopotassium glucarate and glutaric acid from a fermentation broth, the method comprising contacting the fermentation broth with a first antisolvent and then isolating monopotassium glucarate by adjusting the pH of the fermentation broth and antisolvent mixture to about 3.5 through the addition of a sufficient amount of the antisolvent and an acid; and wherein the method further comprises dissolving the isolated monopotassium glucarate in water and acidifying the monopotassium glucarate and water solution using a cation exchange column and adding a second antisolvent wherein the acidified monopotassium glucarate and antisolvent solution is distilled and glutaric acid is isolated. In an embodiment, the first antisolvent is acetone. In an embodiment, the second antisolvent is isopropanol. In an embodiment, the method further comprises the step of isolating the second antisolvent after the distillation of the acidified monopotassium glucarate and antisolvent solution. In an embodiment, the second antisolvent is isopropanol. In an embodiment, the isopropanol recovery yield is greater than 99%. In an embodiment, the addition of the second antisolvent to the acidified monopotassium glucarate solution creates an azeotropic solution. In an embodiment, the method further comprises the step of isolating the first antisolvent after recovering monopotassium glucarate. In an embodiment, the first antisolvent is acetone. In an embodiment, the acetone recovery yield is greater than 99%. In an embodiment, the monopotassium glucarate is recovered with a yield of greater than 99.9%. In an embodiment, the monopotassium glucarate is recovered with a purity of greater than 95%. In an embodiment, the glucaric acid is recovered with a yield of greater than 71%. In an embodiment, glucaric acid is recovered with a purity of greater than 98%. In an embodiment, the energy consumption of the method is less than about 20 MJ/kg for isolating monopotassium glucarate. In an embodiment, the energy consumption of the method is less than about 1460 MJ/kg for isolating glucaric acid.

In an aspect, disclosed herein is a method for isolating a carboxylic acid salt and a carboxylic acid from a fermentation broth, the method comprising contacting the fermentation broth with a first antisolvent and then isolating the carboxylic acid salt by adjusting the pH of the fermentation broth and antisolvent mixture to below 7 through the addition of a sufficient amount of the antisolvent and an acid; and wherein the method further comprises dissolving the isolated carboxylic acid salt in water and acidifying the carboxylic acid salt and water solution using a cation exchange column and adding a second antisolvent wherein the acidified carboxylic acid salt and antisolvent solution is distilled and the carboxylic acid is isolated. In an embodiment, the addition of the second antisolvent to the acidified carboxylic acid salt solution creates an azeotropic solution. In an embodiment, the carboxylic acid salt is recovered with a yield of greater than 99.9% and with a purity of greater than 95%. In an embodiment, the glucaric acid is recovered with a yield of greater than 71% and with a purity of greater than 98%. Other objects, advantages, and novel features of the present invention will become apparent from the following detailed description of the invention when considered in conjunction with the accompanying drawings.

### BRIEF DESCRIPTION OF THE DRAWINGS

FIG. 1 depicts a process flow diagram for producing KGA and GA crystals. Acetone and isopropanol were used as antisolvent for KGA in Antisolvent Crystallization 1 and GA in Antisolvent Crystallization 2, respectively.

FIGS. 2A, 2B, and 2C depict (FIG. 2A) glucaric acid molar speciation at different pH (pKa1=3.17 and pKa2=3.96), (FIG. 2B) glucarate solubility in g/L at various pH in acetone/water mixtures (0, 12, 25 wt. %) at 22° C., (FIG. 2C) KGA from broth mass yield comparison among acid and acetone addition methods at 4° C. The inset photo is the KGA precipitate from broth pH adjusted to 3.5 (left) and broth pH adjusted to 3.5 with acetone 25 wt. % acetone addition (right).

FIG. 3 depicts (FIG. 3A) GA eluent from KGA loading in the DOWEX G26 CEX column. The vertical lines show the cutoff line for GA recovery, (FIG. 3B) Simulated Water-IPA phase diagram using an NRTL model at 50 mbar. The azeotrope formed when the WIPA is 0.875.

FIGS. 4A, and 4B depict process flow diagrams used to estimate energy consumption and determine the ability to recycle the antisolvents in the process for the crystallization and recovery of (FIG. 4A) KGA and (FIG. 4B) GA.

FIGS. 5A and 5B depict a Van′t Hoff plot of (FIG. 5A) KGA in 25 wt. % acetone solution and (FIG. 5B) GA in 87.5 wt. % IPA solution.

FIG. 6 depicts an embodiment of a continuous CEX process consisting of feed loading, cleaning, regeneration, and washing steps.

FIGS. 7A and 7B depict (FIG. 7A) equilibrium of glucaric acid (GA) and lactones, (FIG. 7B) Simulation of GA equilibration at 300K based on rate constants given in Table 6 depicts.

FIG. 8 depicts a TGA calibration curve for GA solutions.

FIG. 9 depicts solubility of potassium glucarate (KGA) in a solution of acetonitrile, acetone, or ethanol with varied water content at 4° C.

FIGS. 10A, 10B, 10C, 10D, and 10E depict (FIG. 10A) precipitation of KGA from broth using acid addition (left) and acid and acetone addition method (right), (FIG. 10B) recovered KGA obtained from acid addition, (FIG. 10C) recovered KGA from acid and acetone addition method, (FIG. 10D) recovered GA via antisolvent crystallization method, (FIG. 10E) Optical microscopy image of GA (×200 magnification).

FIGS. 11A and 11B depict (FIG. 11A) simulated water-IPA phase diagram using an NRTL model at 50 mbar. The azeotrope formed when the WIPA is 0.875. (FIG. 11B) Comparison of antisolvent effect of IPA and acetonitrile (ACN) on GA solubility at 4° C.

FIGS. 12A and 12B depict (FIG. 12A) FTIR and (FIG. 12B) 1H-NMR spectra of the recovered products: KGA, unwashed GA, GA after an acetone wash.

FIG. 13 depicts 1H NMR spectrum for KGA 1H NMR (300 MHZ, D₂O) δ 4.27 (d, J=3.1 Hz, 1H), 4.22 (d, J=4.7 Hz, 1H), 4.170 (dd, J=3.1, 5.7 Hz, 1H), 3.95 (t, J=4.9 Hz, 1H).

FIG. 14 depicts 1H NMR spectrum for unwashed GA showing presence of lactone impurities. The following multiplet report is for the GA molecule only: 1H NMR (300 MHZ, D₂O) δ 4.49 (d, J=3.1 Hz, 1H), 4.38 (d, J=5.0 Hz, 1H), 4.17 (dd, J=3.1, 5.7 Hz, 1H), 3.99 (q, J=3.6 Hz, 1H).

FIG. 15 depicts ¹H NMR spectrum for washed GA. ¹H NMR (300 MHZ, D₂O) δ 4.45 (d, J=3.1 Hz, 1H), 4.35 (d, J=4.9 Hz, 1H), 4.17 (q, J=2.9 Hz, 1H), 3.98 (q, J=3.5 Hz, 1H).

FIG. 16 depicts XRD pattern of KGA (upper) and GA (lower).

FIGS. 17A and 17B depict DSC curves of (FIG. 17A) KGA and (FIG. 17B) GA,

FIGS. 18A and 18B depict an Aspen Plus model for the crystallization and solvent recovery of (FIG. 18A) KGA and (FIG. 18B) GA.

FIG. 19 depicts KGA (0.06 M) breakthrough curve compared with a rate model simulation.

### DETAILED DESCRIPTION

Biocatalysis offers high selectivity, mild reaction conditions, and the ability to effectively convert renewable sugars to platform chemicals for fuels, plastics, and other renewable chemicals. These approaches also align with ‘green chemistry’ principles, having the potential to minimize waste streams, eliminate heavy metal catalysts, and increase energy efficiency. GA is an example of a promising platform carboxylic acid that can be produced via fermentation with several green chemistry benefits over traditional catalytic oxidation processes. Notably, in the biological process, fermentation occurs under mild conditions (30° C. and pH 7.0) without generating excess amounts of toxic oxidants or requiring high pressure reactors. However, the isolation of GA from fermentation broth remains challenging and there has been little work on improving a separation process.

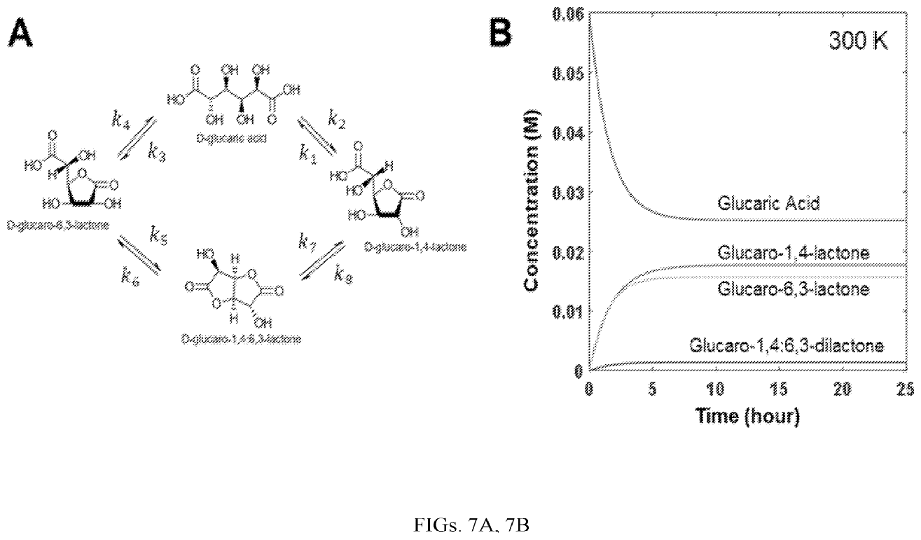

Methods to recover GA diacid crystals are seldom reported due to challenges in avoiding lactonization reactions during the separation process. GA isomers exist stably in the mono- or di-salt form with counter cations such as potassium or sodium. We distinguish the monopotassium glucarate (KGA) salt from the di-potassium salt (K₂GA), and the diacid (GA) to identify the form of the final crystalline products. Further, the GA diacid is readily lactonized into D-glucaro-1,4-lactone, D-glucaro-6,3-lactone, and D-glucaro-1,4:6,3-dilactone under aqueous conditions, as shown in FIG. 7A. Brown et al. calculated the equilibrium and rate constants for each lactonization reaction in FIG. 7A based on NMR data. Using those data, we found approximately 55% of GA is lactonized within 6 hours, even at 30° C. (FIG. 7B). This rapid lactonization complicates the development of a method to isolate purified GA crystals.

However, Armstrong et al. reported a method to produce crystalline GA from the monopotassium glucarate salt (KGA) via cation exchange (CEX) and azeotropic drying using a water-acetonitrile (ACN) system. In that system, the addition of ACN formed an azeotrope with water allowing low temperature water removal to minimize GA lactonization. Although the ACN-water system achieved high purity (>99%) and a high recovery yield (98.7%) of crystalline GA, the feed concentration was low (5 g/L KGA) with an overall diluted reaction solution (95:5 ACN: GA aqueous solution v/v), limiting the method's efficiency and scalability. Specifically, the starting GA solution volume is increased 19× due to the large amount of ACN needed to azeotropically remove the water. This 19× volume increase in the stream requires large crystallization tanks and a large amount of ACN solvent recovery. This results in a large energy consumption per product for the post-crystallization ACN recovery process. Accordingly, developing a more sustainable and scalable processes to recover specific purified forms of KGA and GA from fermentation broth is a key challenge to improving the economics of bio-glucaric acid and its ultimate commercialization.

To address the need for optimized downstream processing routes for GA, the methods, systems and compositions of matter disclosed herein proposes a scalable, environmentally friendly, and economically feasible antisolvent separation process for the recovery of GA and its salts from fermentation broth. Antisolvent crystallization involves combining the product solution with another solvent in which the product is only slightly soluble. This significantly reduces the solubility of the product in that solution, allowing it to be recovered as a precipitate.

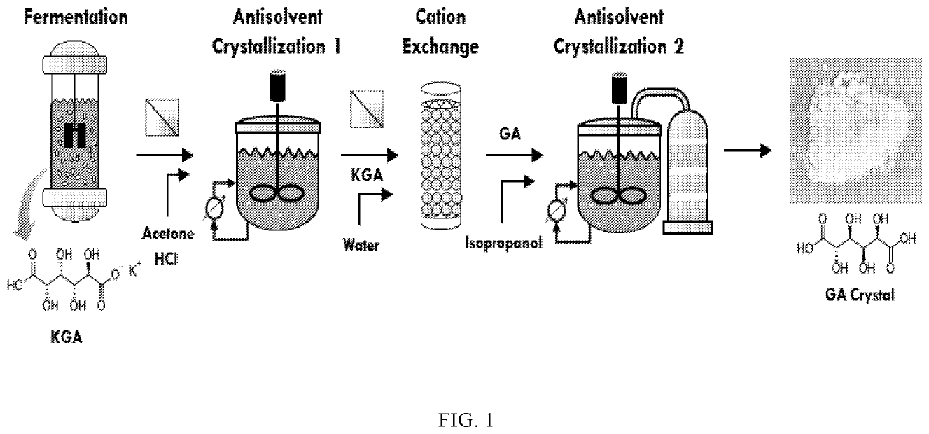

The separation processes are described in FIG. 1. First, dipotassium glucarate (K2GA) was produced via fermentation at a neutral pH to generate a broth. Solid KGA is then recovered from the broth by employing 1) pH-adjustment from 7 to 3.5 to generate KGA, 2) antisolvent crystallization of KGA using acetone at an acetone-to-water mass ratio of 1 to 2.95, 3) KGA product filtration, and finally 4) acetone antisolvent recycling via distillation of the supernatant. Next, crystalline GA was produced from the purified KGA via another antisolvent crystallization process, which consists of the following steps: 1) cation exchange for acidification and K+ removal, 2) IPA antisolvent crystallization of GA, 3) GA crystal recovery by azeotropic drying, and 4) IPA antisolvent recycling. The physicochemical and thermodynamic properties of the purified KGA and GA products were analyzed and used to develop Aspen Plus models for solvent recovery, which enable calculation of the energy consumption on the downstream process. The proposed route to separate GA uses IPA which acts as both an antisolvent and, concomitantly, an azeotropic drying aid. This results in reducing the antisolvent amount by 2.1 times over the ACN-water system, which acts as a mild antisolvent and an azeotropic drying aid. The antisolvent crystallization process proposed in this work is could also be applicable to the purification of other oxidation products from glucose, such as gluconic acid and mevalonic acid.

Glucaric acid is regarded as a top-value added compound and thus it is widely studied for its synthetic routes from glucose and other renewable feedstocks. However, due to prevalent lactonization, the recovery of purified glucaric acid from fermentation broth is challenging. Accordingly, an efficient method for glucaric acid separation, especially its diacid form, is necessary to facilitate its utilization in various applications. A robust separation process that produces glucaric acid crystals from fermentation broth is disclosed herein. This process first recovers purified monopotassium glucarate from broth and then recovers purified glucaric acid through acidification and antisolvent crystallization. Isopropanol was found to be an effective antisolvent reducing the solubility of glucaric acid while concomitantly forming an azeotrope with water. This allows solvent removal at low temperature through azeotropic drying, which avoids lactonization, and thus prevents impurities in the resulting crystals. Overall, this process was found to separate monopotassium glucarate and glucaric acid with a recovery yield of >99.9% and 71% at purities of c.a. 95.6 and 98.3% respectively. Process modeling demonstrates the ability to recycle the antisolvents IPA and acetone with >99% recovery and determined the energy consumption to be ˜20 MJ/kg for isolation of potassium glucarate and 1,456 MJ/kg for glucaric acid. The approach detailed in this work is applicable to the separation of other highly oxygenated bio-carboxylic acids (e.g., mevalonic acid) from fermentation broths, as well as to their recovery from abiotic reaction solutions.

### Experimental

Materials: Glucaric acid fermentation broth was obtained from Kalion, Inc. The final titer of glucaric acid was c.a. 69.5 g/L measured by LC analysis. Broth was sterile filtered through a 0.2 μm ceramic filter before any downstream processing. Cation exchange was carried out on DOWEX G-26 H ion exchange resin (DuPont) packed into a Cytiva XK 16/20 chromatography column. 6 M HCl (Carolina Biological) was diluted to 1 M for acidification of broth and regeneration of the DOWEX G-26 resin. Acetone (VWR, >99.5%), was used for antisolvent of KGA and 2-propanol (Sigma-Aldrich, >99.5%) was used as an antisolvent and azeotropic drying aid to recover crystalline GA. All water used was ultra-high purity (>17.2 MS2-cm). D₂O with 0.05 wt. % 3-(trimethylsilyl) propionic-2,2,3,3-d4 acid was purchased from Sigma-Aldrich for NMR analysis.

KGA Solubility Test: KGA solubility was quantified by adding excess KGA to UHP water and adjusting pH between 1-6 by addition of 2 M HCl or 2 M KOH with stirring. The samples were mixed, allowed to sit overnight, and then the pH was measured again. The samples were filtered, diluted with a pH 7 phosphate buffer to reduce lactonization and the KGA concentration was quantified by LC. Similarly, KGA solubility in a 25.2 wt. % acetone solution was measured by preparing KGA-saturated solution samples at −20° C., 22° C. and 40° C. and quantified by using the liquid chromatography (LC) method.

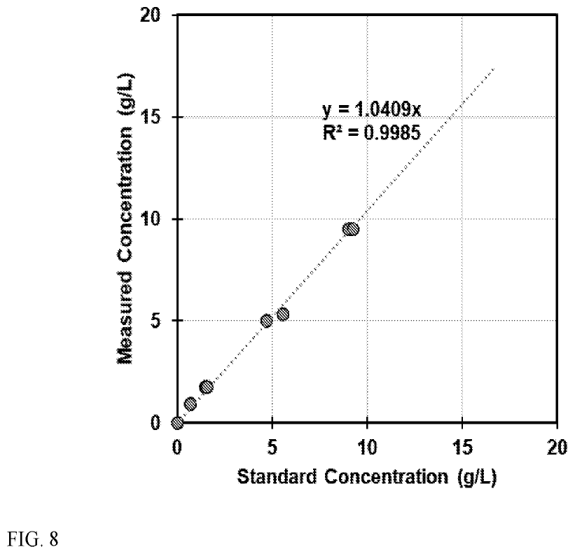

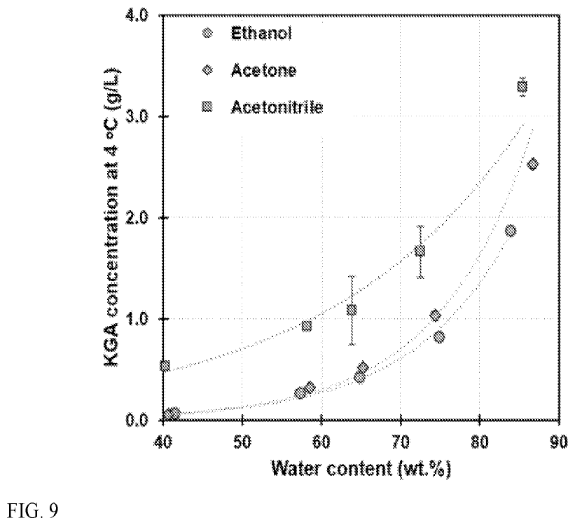

GA Solubility Test: Due to the lactonization potential of glucaric acid, the solubility was estimated using thermogravimetric analysis (TGA) based on the mass balance of the saturated solution. A sample of acetone-washed GA was dissolved in 3 mL of the IPA/water mixture (7:1 by mass) to excess, sonicated, and allowed to settle overnight at each of three temperatures: −20° C., 4° C. and 22° C. Next, the solutions were filtered at a pore size of 0.45 μm. These saturated solutions were dropped into an aluminum Differential Scanning calorimetry (DSC) pan and placed on dry ice to partially freeze them and slow the evaporation rate so that the mass of the total solution could be measured by TGA. The partially frozen solution was heated from room temperature at 5° C. per minute to 110° C. and held isothermally for 15 minutes to allow the solvent to evaporate. In this way, the mass of the remaining species in the pan was determined to μg precision. This was taken to be the total mass of GA that had dissolved in the solvent, including possible lactone products, in a catch-all method. The method was tested for known concentrations in the range of 1 g/L to 15 g/L with a blinded relative error of approximately 6%, compared to 15% for the LC method used for KGA analysis (see FIG. 8).

Producing KGA from fermentation broth: For the recovery of KGA, acetone was used as the antisolvent as it provided the lowest glucarate solubility in organic-aqueous systems. 2 M HCl (100 mL) was added to a broth solution (500 mL) to adjust the pH to 3.5. Acetone (250 mL) was slowly added into the solution with stirring. The broth mixture was cooled to 4° C. for 24 hours to promote crystal growth and increase yield, then KGA crystals were recovered via vacuum filtration. The filtered product was mixed with a 50% acetone/water solution and filtered three times to completely remove any leftover broth solution. The recovered crystals were dried in a vacuum oven until constant mass was achieved. Purity of the crystalline KGA was evaluated using liquid chromatography (LC), FTIR, DSC and ¹H NMR.

Glucarate acidification via cation exchange (CEX): To generate crystalline GA, ion exchange of KGA in water was performed using DOWEX G-26 resin. This resin was pretreated by covering ˜15 g of dry resin with 1 M HCl. The resin was slurry packed into a GE XK 16/20 column and then connected to a Cytiva ÄKTA Pure Chromatography system. The packed column size was 16 mm in inner diameter and 10 cm in length. This allowed for continuous pH, conductivity, and temperature monitoring. The resin was rinsed with 7-10 BV of UHP water at a flow rate of 4 mL/min until a neutral pH was achieved. KGA purified from fermentation broth was dissolved in water. Here, 1 M KOH was added to increase the pH and solubility to yield a concentration of 0.1 M KGA. Effluent was collected as waste until the pH of the effluent dropped below 2.5 and stabilized, which indicated that the effluent pH was below the pKa of GA and that the GA concentration was constant. Additionally, UV 190 nm, 210 nm, and conductivity readings were steady in this range. This effluent fraction was collected into a clean beaker and placed in an ice bath to inhibit lactonization of free GA in solution. After the desired volume of KGA solution had been pumped into the column, UHP water was added to prevent the column from running dry. The effluent was then collected until the pH was above 2.5 and UV 190 nm signal began to decrease. UHP water was pumped through the column at a rate of 3 mL/min until the effluent was a neutral pH. Then, the column was washed with UHP water to remove salts in the column and regenerated by loading 1 M HCl. When the pH curve showed a breakthrough of HCl, the column was washed with water until the conductivity dropped to below 0.01 mS/cm.

Antisolvent crystallization of GA with azeotropic drying: For the recovery of GA, 2-propanol (IPA) was used as the antisolvent and azeotropic drying aid. IPA was added to the GA solution at a mass ratio of 7:1 (12.5 wt. % aqueous solution) to create a low-boiling azeotropic solution. GA was recovered by reducing volume of the azeotrope using rotary evaporation to create a supersaturated solution of GA. This mixture was gradually evaporated using a Buchi Rotavapor® R-300 Rotary Evaporator at 30 mbar and 22° C. to one tenth the original volume. At this point, small seed crystals of GA formed throughout the solution. When the solution was not in the rotary evaporator, it was kept on ice to slow lactonization. The concentrated GA solution was stored overnight in a −20° C. freezer to further crystal growth from the seed crystals. Next, the GA crystals were recovered via rotary evaporation. Any lactones present in the GA crystals were removed by washing two times with an excess amount of acetone and the remaining crystals were then vacuum filtered. Finally, the GA crystals were vacuum dried (70° C., 22 mmHg) for six hours.

Solvent recovery model: An Aspen Plus model was built and optimized to estimate the energy footprint of solvent recycling via distillation. This model was developed to recover the antisolvents, acetone for KGA and IPA for GA recovery, respectively, after the crystalline products were separated. The process was optimized to achieve a >99% recovery yield for both product crystal as well as the antisolvent with >99.0% purity. The simulation was split into two sections: 1) crystallization, and 2) solvent recovery. In the crystallization section, KGA and GA were input as user-defined components, and the UNIFAC method was chosen because of its reliable predictions based on functional group contributions. NRTL was used for the solvent recovery section to accurately simulate the distillation process.

DSC for purity analysis: Modulated differential scanning calorimetry (MDSC) was used to measure the purities of crystalline glucaric acid and potassium glucarate via melting point depression. Glucaric acid samples were tested from 20 to 140° C. at a ramp rate of 2° C./min with a modulation amplitude of 1° C. and a modulation period of 60 seconds, and potassium glucarate from 20 to 190° C. with the same modulation.

Liquid chromatography (LC) for KGA analysis: The concentration of glucaric acid in aqueous solutions was quantified using an Agilent 1290 Infinity Series LC system equipped with UV-diode array detection at 210 nm. 15 μl samples were injected into a Phenomenex Luna C18(2) 5 μm, 150 mm×4.6 mm column at a temperature of 35° C. An isocratic mobile phase of 20 mM potassium phosphate at pH 7 was pumped at a flow rate of 0.65 mL/min for 7 min.

FT-IR analysis Crystalline glucaric acid and crystalline potassium glucarate were analyzed via FTIR on a Nicolet iS50 spectrometer in ATR mode. Spectra were measured from 450-4000 cm⁻¹ at 2 cm⁻¹ resolution over 24 scans.

NMR analysis for GA analysis: Structural and purity analysis was performed using a Varian 300 MHz NMR Spectrometer. For all analyses, samples were prepared at 2-4 mg/ml using D₂O with 0.05 wt % 3-(trimethylsilyl) propionic-2,2,3,3-d4 acid. All spectra were referenced from D₂O and processed using Bruker TopSpin.

### Results

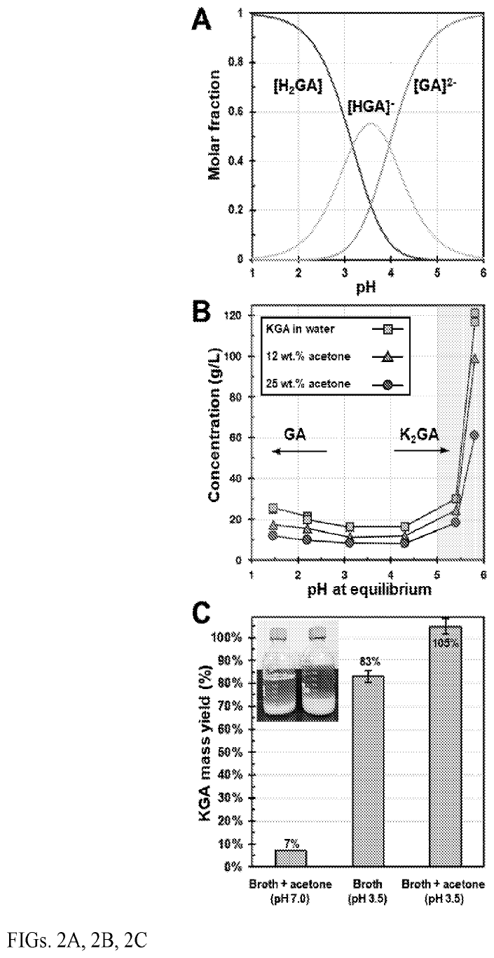

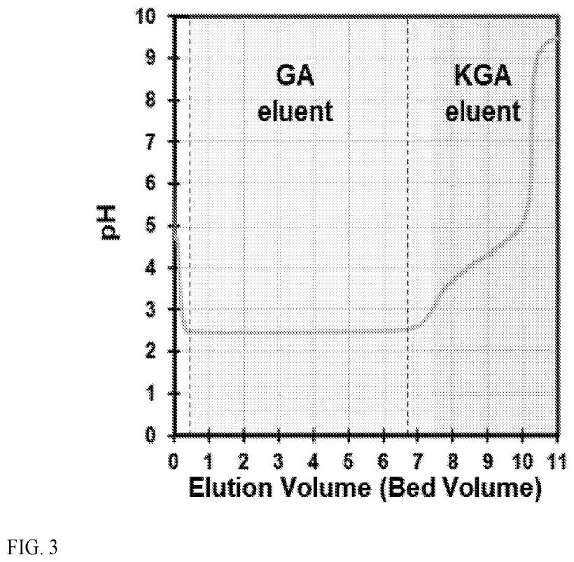

GA production from fermentation broth: Cells and debris were removed using 0.2 μm filtration. After filtration, the broth permeate pH remained at 7.0 such that all the produced GA stably existed as dipotassium glucarate (K₂GA) as shown in FIG. 2A and thus did not form any lactone species.

Solubility of glucarate at various pH's in antisolvents: The solubility of different glucarate forms was measured in the pH range of 1 to 6 in water and in water-antisolvent systems to determine an optimal condition for separation from aqueous fermentation broth. Previous work has shown the solubility of mono potassium glucarate (KGA) is lower than the dipotassium form and the diacid form (GA), and, therefore, KGA can be precipitated by adjusting the pH to 3.4 where the KGA form is present in the highest concentration. This speciation as a function of pH is seen in FIG. 2A where the molar fraction of each form is calculated from the reported pKa values of glucaric acid (pKa1=3.17 and pKa2=3.96). Here KGA reaches a maximum concentration of 55 mol % at pH 3.6. However, the KGA yields cannot reach 100% by pH adjustment alone. Accordingly, we added an antisolvent to increase the KGA yield. Preliminary antisolvent screening results found acetone and ethanol (FIG. 9) to be promising antisolvents since both have low dielectric constants that reduce the KGA solubility. Acetone was chosen as the preferred antisolvent because of its low boiling point and no azeotrope formation with water, which is advantageous for solvent recycling via distillation.

FIG. 2B shows the solubility of glucarate in the pH range of 1 to 6 and with acetone concentrations ranging from 0-30 wt. %. When the pH is close to neutral, glucarate is approximately 100 mol % K2GA, which has a high solubility in water (>120 g/L). As the pH is decreased to 3.5, KGA dominates the mol fraction at 55 mol % and KGA's solubility is around 16 g/L (FIG. 2A), which is significantly reduced to 13.5 mol % of the solubility of K2GA at pH 5.8. Further, an additional 50% reduction of the KGA solubility (down to ˜8.1 g/L) was observed by adding acetone (25 wt. %). Thus, the lowest solubility was observed between a pH of 3.1 and 4.3 where KGA− is largely formed, in this pH range 83% of the glucarate species present at pH 5.8 precipitates as KGA due to its low solubility (FIG. 2C). As the pH is decreased further toward 1.5 the solubility increases by about 55% (26 g/L), here KGA is fully protonated to H2GA and stabilized via hydrogen bonding interactions with water.

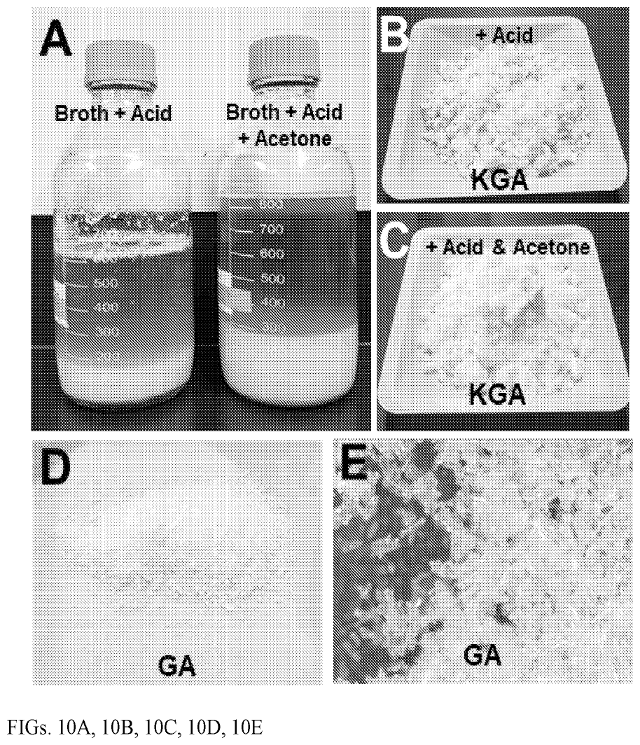

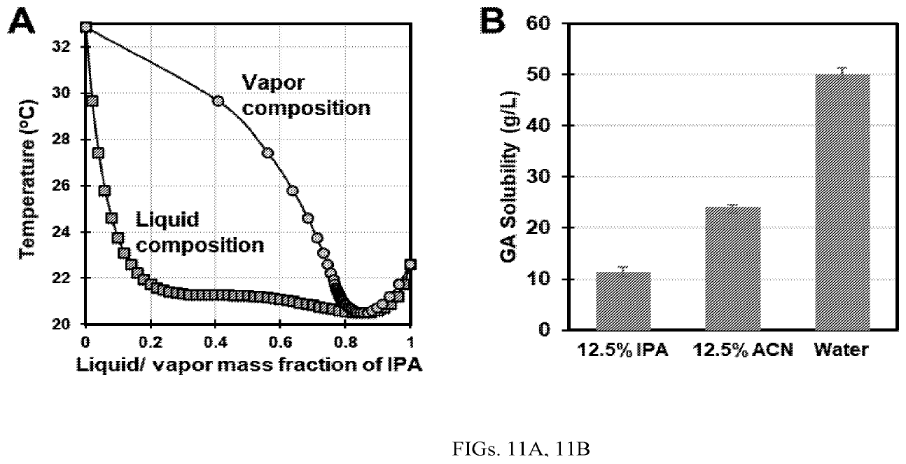

KGA recovery from fermentation broth via antisolvent crystallization: Using the solubility results, KGA was recovered from the filtered broth by adjusting the pH and with simultaneous addition of 25 wt. % acetone. FIG. 2C compares the KGA recovery yield with these methods. Adding only 25 wt. % acetone into the broth without pH adjustment (note the starting broth pH was 7.0) caused phase separation between the acetone-rich phase and the salt-rich phase resulting in a KGA recovery yield of only 7%. By adjusting the pH to 3.5, without acetone addition, the KGA recovery yield was 83%. Combining these two methods by adding 25 wt. % acetone in the pH adjusted broth (pH=3.5) resulted in essentially quantitative recovery of KGA. As seen in the inset photo in FIG. 2C and FIG. 10, the amount of precipitated KGA from this pH adjustment plus acetone addition method is visually larger compared to that from the acid addition method and the acetone addition method. Although the solubility of KGA was not expected to be zero at this condition (measured as around 8.1 g/L from control experiments in FIG. 2B), almost all the KGA precipitated out. This could be the result of the lower equilibration temperature and the ion effect by other salt species in the broth that could further reduce the KGA solubility.

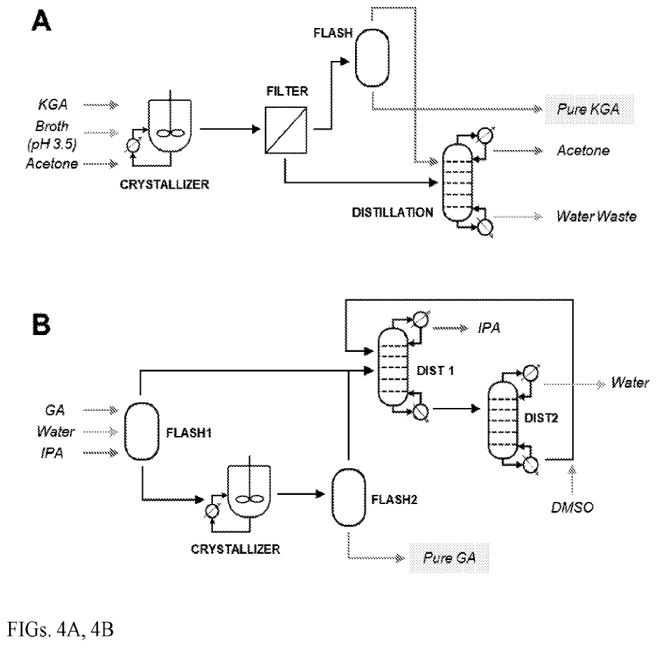

GA recovery via CEX and antisolvent crystallization: To recover the free acid form of glucaric acid, the purified KGA was redissolved in water at a concentration of 0.1 M and treated with a cation exchange (CEX) process to exchange the K+ cations to H+ cations. Since the selectivity of K+ over H+ is known (2.54) in polystyrene-based sulfonic resins with 10% cross-linkage, K+ cations were easily adsorbed to the resin by exchanging with preloaded H+ cations.30 The GA-anions were not adsorbed during the elution. when measuring the breakthrough curve, the GA free acid was eluted without adsorption, and the eluent pH dropped immediately, remaining constant at ˜2.5 until the column was fully saturated with K+ cations (FIG. 3). The eluent pH increased after saturating the resin with K+ cations. Saturation occurred at approximately 7 bed volumes of KGA solution (0.1 M, pH adjusted by KOH), which indicates the column capacity of c.a. 2.0 mequiv/ml. The CEX process was further tested using several KGA solutions to optimize the loading conditions.

As disclosed herein, KGA was dissolved in high purity water near the saturation limit (0.06 M) and a higher feed concentration (0.1 M) was achieved by adjusting pH to 9.4 with additional KOH. We used the 0.1 M feed condition because the total mass of GA produced for a given loaded volume of KGA solution increased with similar yields and purities compared to 0.06 M condition (discussed below). Also, seed crystal formation and growth are a concentration-driven process, and those rates in 0.1 M concentrations are therefore faster than at 0.06 M, which is favorable in a large-scale process. We also attempted to use higher feed concentrations of KGA at 0.24 M by adding KOH to increase the KGA solubility. However, K+ cations were not fully exchanged leading to the elution of some KGA, and this prevents downstream GA crystallization. Thus, it is not recommended to increase the KGA concentration above 0.1 M in the feed to the CEX resin.

To recover crystallized GA, the GA solution was collected when the eluent pH was constant at 2.5 (FIG. 3), cooled on ice to reduce room temperature lactonization, and mixed with IPA at a 7:1 mass ratio of IPA to GA to recover crystalline GA via azeotropic distillation. Low temperature solvent removal is required to mitigate glucaric acid from lactonizing to form d-glucaro-1,4-lactone, d-glucaro-6,3-lactone, and d-glucaro-1,4:6,3-dilactone, which exist in equilibrium with glucaric acid. Furthermore, GA has a 56% higher solubility than KGA, even in water/antisolvent mixtures as compared in FIG. 2B. Therefore, the selection of an antisolvent that also acts as an azeotropic distillation aid is critical for GA recovery. To that end, we selected IPA, because it forms a low boiling azeotrope with water of 21.0° C. at 50 mbar and requires a relatively low solvent-to-water mass ratio of 7:1 based on a NRTL model. Furthermore, GA has a lower solubility in IPA than in acetonitrile. Specifically, the solubility of GA in IPA-water is 53% lower than that in ACN-water, and 77% lower than that in water. The addition of IPA to the aqueous GA solution therefore generates a concomitant antisolvent effect accelerating the formation of GA crystals while also allowing low temperature water removal.

The IPA antisolvent crystallization process was carried out in three steps: (1) concentration, (2) seed growth, and (3) complete solvent removal. First, the GA solution-IPA mixture was concentrated 10-fold by rotary evaporation (30 mbar, 23° C.). We observed that the evaporation rate was nearly two times faster at 30 mbar than 50 mbar. Accordingly, 30 mbar was used to lower the processing time as a means to prevent lactonization.

Table 2 presents the overall yield of this three step GA crystallization process based on the initial concentration of the KGA solution that was fed into the CEX column. GA recovery yields and purities were very similar between 0.06 M and 0.1 M KGA feed solution. The yield loss was mainly due to transfer losses of lactones that were stuck on the wall of the vial. After washing the recovered crystals with acetone, the recovery yield decreased to 71.1% but the GA purity was increased to 98.3% as determined by DSC.

TABLE 2
KGA feed conditions in the GA crystallization process and the
resulting yield and purities. The GA yield was determined by
a weight ratio (mGA/mKGA) and the purity by DSC.

| Feed | GA Yield | GA Purity |
|---|---|---|
| concentration | (%) | (%) |
| 0.06 M KGA | 93.0 | 88.6 |
| 0.1 M KGA | 92.5 | 88.7 |
| (pH 10) | | |
| 0.1 M KGA | 71.1 | 98.3 |
| (Washed) | | |

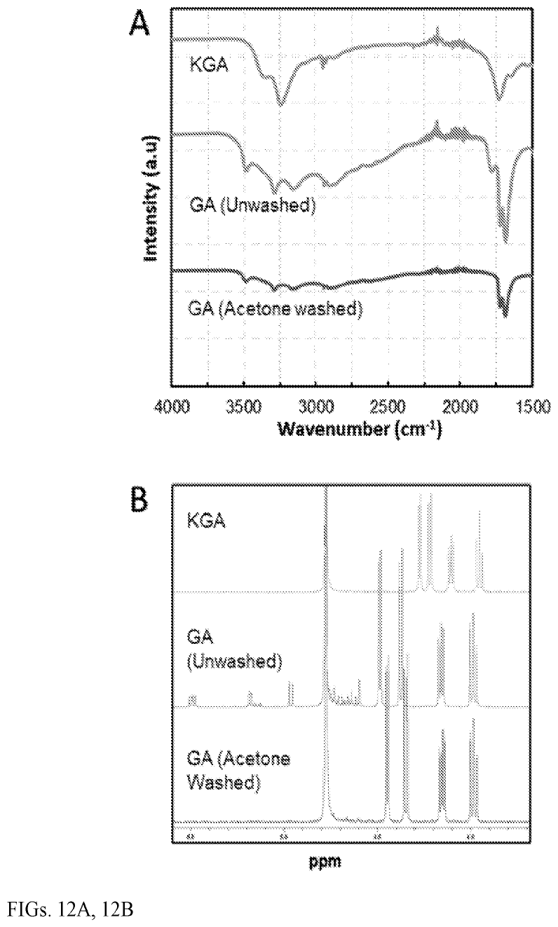

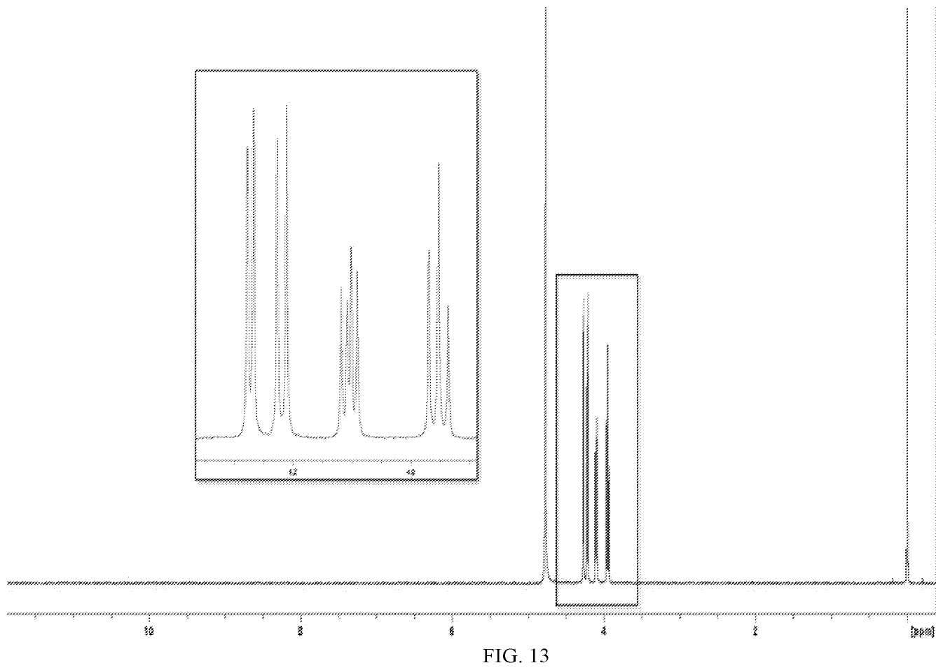

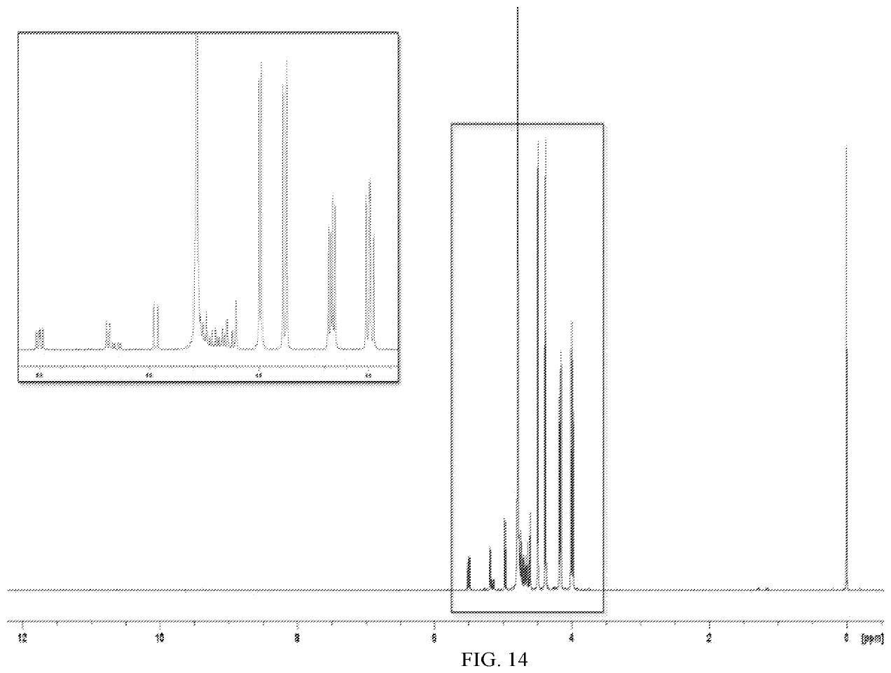

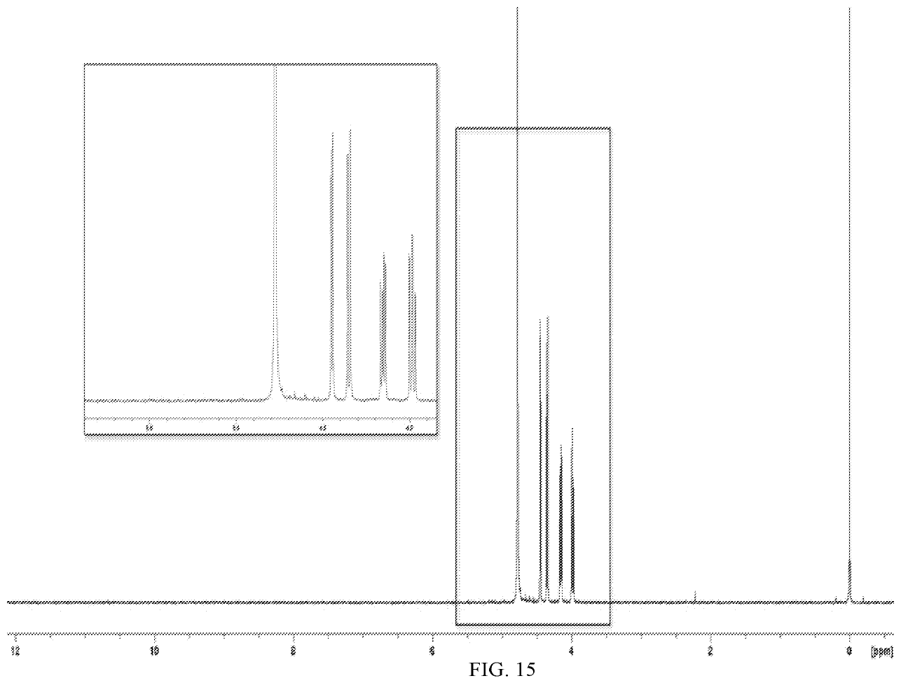

To determine the source of the impurities, the recovered KGA and GA products were characterized with ¹H NMR and ATR-FTIR. The 1H-NMR spectrum of crystalline KGA revealed a highly pure product (FIG. 12B, green trace). Conversion of GA from KGA was clearly observed, with a downfield shift of the terminal protons from 4.21 ppm and 4.26 ppm to 4.36 ppm and 4.48 ppm, but with the presence of the undesired lactone species downfield (FIG. 12B, red trace). The presence of lactones was qualitatively assessed by the δ-lactone peak at 1775 cm⁻¹. The intensity of this peak was significantly reduced after washing the crystals with acetone with 1 to 1 solid to liquid volume ratio, and completely eliminated after two successive acetone washes, which was also confirmed by ¹NMR (FIG. 12B, blue trace). Notably, the FTIR spectrum of GA obtained in this study matches with that reported by Armstrong, et. al, though without the presence of acetonitrile C≡N stretching around 2250 cm⁻¹. Further, these observations were consistent with other previously reported data.

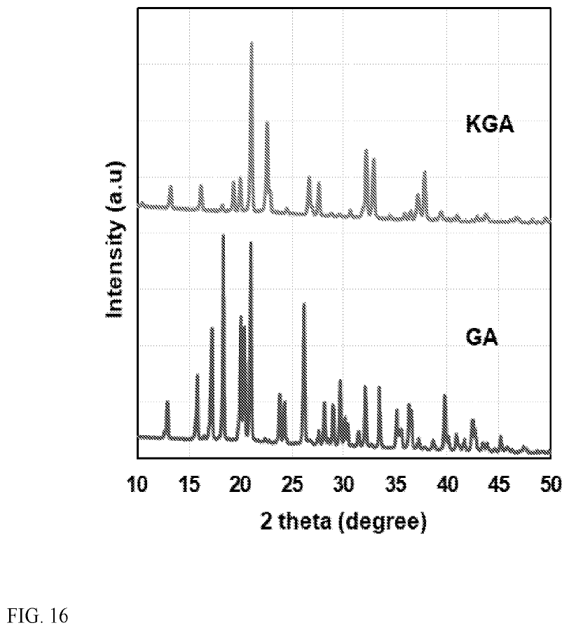

The XRD patterns for KGA and GA were also measured. Different peaks between KGA and GA indicate variances in crystal structures (FIG. 16). The XRD pattern of GA is consistent with previously reported data. Optical microscopy images of the GA crystal revealed a monoclinic structure with around 200 μm on the longest axis as expected from XRD pattern (FIG. 10E).

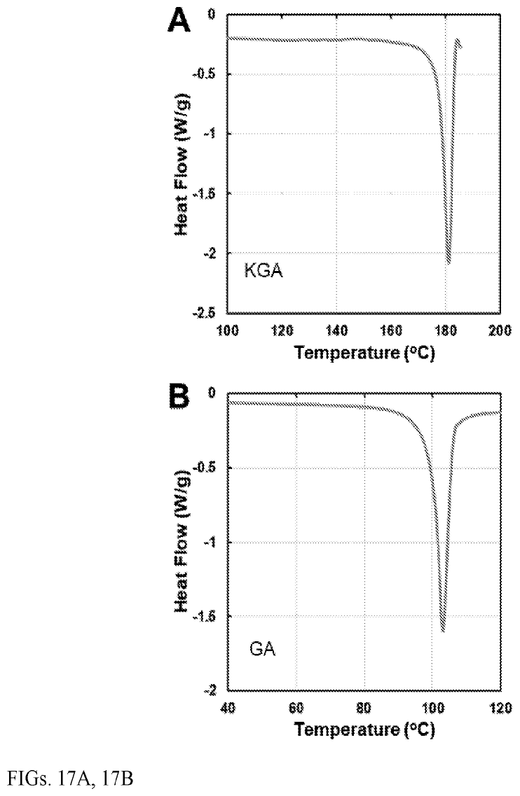

DSC analysis showed that the melting point of KGA and GA was 182.5° C. and 105° C., respectively (FIG. 17). Additionally, the purities of KGA and GA were measured with DSC by using the melting point depression method (Table 2). DSC is a highly accurate method to calculate absolute purity and it is possible that some impurities cannot be detected in NMR, which only shows the ¹H resonance. The DSC measured purities of KGA and GA were 95.6% and 98.3%, respectively.

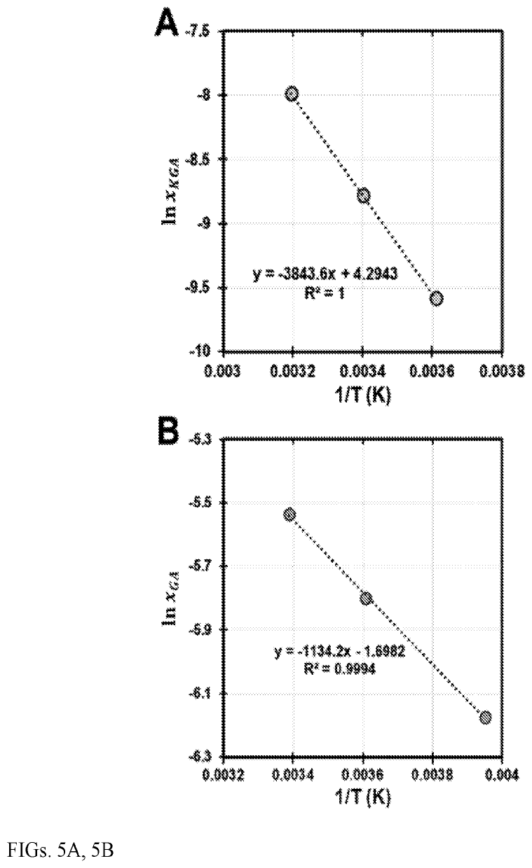

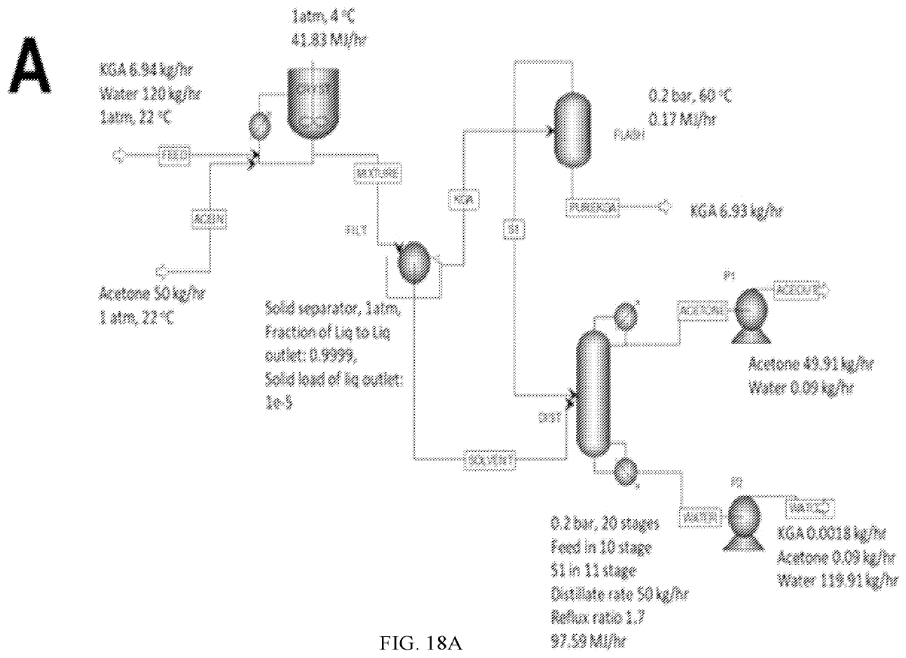

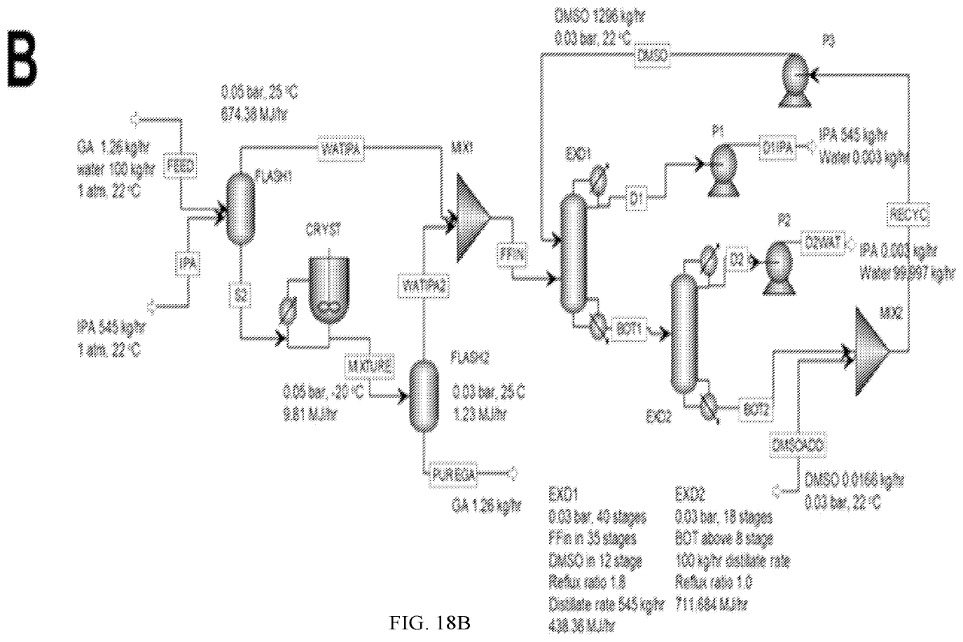

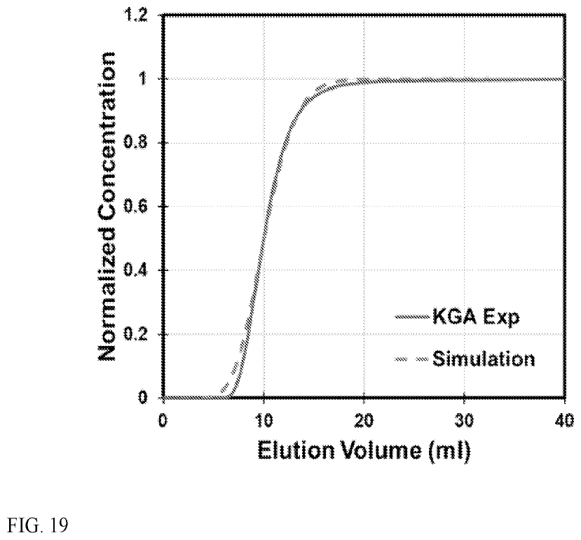

Energy Consumption for Solvent Recovery. Aspen Plus models of both the KGA and GA downstream processes were developed to determine the energy consumption on a kg product basis and determine the ability to recycle the antisolvents. Process flow diagrams are shown in FIG. 4. To simulate the thermodynamic properties of KGA and GA, a UNIFAC model was employed, and their thermodynamic properties such as heat capacity, solubility, and solid molar volume were obtained either from product characterization data from this work or from literature data (FIG. 18 and Table 7). The feed concentration of KGA and GA from the analysis of the broth was used as the simulation input stream. However, other salts and impurities in the fermentation broth were not considered and were assumed to remain in the wastewater, negligibly affecting the thermodynamic properties of KGA and GA in all processes. For the KGA recovery process, pH adjusted fermentation broth (pH=3.5) was fed into the crystallization vessel with acetone as the antisolvent at 25 wt. % and a filtration process was modeled to recover solid KGA (FIG. 4A). Then, pure acetone was recovered from the supernatant by distillation, and recycled as the KGA antisolvent. The resulting water stream that includes non-quantified fermentation broth compounds was sent to wastewater treatment. Overall, both KGA and acetone were both recovered with >99% yield and >99% purity. The heat duty for acetone recycling in the KGA purification process was determined to be 20.12 MJ/kg KGA as shown in Table 3 Approximately 35% of net heat duty was used for crystallization, while the other 65% was used for acetone recovery.

The modeled GA purification process includes crystallization and IPA recycling by distillation to isolate GA from the GA eluent after CEX (FIG. 4B). IPA was added to the GA CEX eluent at a mass ratio of 7 to 1 to generate the azeotropic mixture. The solution was then evaporated in Flash 1 at 25° C. and 50 mbar to concentrate the GA 10-fold. This concentrated solution was then sent to a seed crystallization tank at −20° C. to form seed crystals. The seed solution was then sent to Flash 2 (30 mbar, 25° C.) where IPA and water were removed in the overhead to recover solid GA in the bottoms. The overhead of Flash 2 containing the azeotrope of water and IPA was combined with the overhead of Flash 1 and then separated into pure water and pure IPA with an extractive distillation process using DMSO as an entrainer. Since both separated IPA and water were highly pure (>99.0%), they can be recycled into the KGA redissolution or GA antisolvent crystallization process. When using the same feed concentration as used experimentally (0.06 M) and assuming a 100% GA recovery yield with no lactonization side-products, the net heat duty required for the overall process was determined to be 1,456.56 MJ/kg GA (Table 3). Overall, the energy consumption for GA purification was much higher than that of KGA purification, with the solvent evaporation and recovery processes consuming ˜38% and ˜63% of net heat duty, respectively. For an energy-efficient approach, an optimized heat integration process should be applied to reduce net heat duty on the solvent recovery process.

TABLE 3
Heat duty associated with each unit operation in FIG. 5

| Unit | Heat Duty (MJ/kg product) | |
|---|---|---|
| Operation | KGA | GA |
| Crystallizer | 6.03 | 7.79 |
| Flash | 0.02 | Flash 1 = 535.22 |
| | | Flash 2 = 0.98 |
| Distillation | 14.07 | Dist 1 = 347.90 |
| | | Dist 2 = 564.67 |
| Net heat | 20.12 | 1456.56 |
| duty | | |

### Discussion

The enthalpy and entropy of dissolution of KGA and GA. The enthalpy and entropy of dissolution are calculated here using the solubility of KGA and GA in solutions of identical solvent composition as a function of temperature. The solubility of KGA and GA and related thermodynamic properties (enthalpy and entropy of dissolution) are important because they can be used for building a crystallization model and optimizing crystallization conditions in a large scale process. Accordingly, using the solubility data obtained for each compound (FIG. 2B), the enthalpy and entropy of dissolution of both KGA and GA were calculated using the Van't Hoff equation.

$$ln x = - \frac{{Δ}_{dis} H}{R T} + \frac{{Δ}_{d i s} S}{R} \qquad ( 1 )$$

In Equation 1, x is the mole fraction of a compound in the solvent, Δ_dis H and Δ_dis S are the enthalpy and the entropy of dissolution, T is the absolute temperature, and R represents the ideal gas constant. The solubility of KGA or GA was measured by varying temperature to plot ln x versus 1/T, and the resulting values of enthalpy and entropy of dissolution was determined from the slope and the intercept, respectively.

FIG. 5 displays the Van't Hoff plot for KGA in a water/acetone mixture and GA in a water-IPA mixture, respectively. The antisolvent loading in each mixture is the same as used in each purification process. Table 4 provides the dissolution enthalpy and entropy of both KGA and GA calculated from Eq. (1) using the linear fits in FIG. 5. The positive values of enthalpy for both indicates that the dissolution reaction of both compounds is endothermic in the experimental temperature range. The slope of GA solubility data yields a negative entropy of dissolution for GA. This could be because dissolved GA is in equilibrium with lactones, and they form dimers or organized structures that are represented by this value.

TABLE 4
Parameters for calculating enthalpy of
dissolution of KGA and GA.

| | | AdisH | AdisS |
|---|---|---|---|
| Compound | Solvent | (KJ/mol) | (J/mol/K) |
| KGA | Acetone | 31.96 | 35.70 |
| | 24.7 wt. % | | |
| GA | Isopropanol | 3.68 | −40.78 |
| | 87.5 wt. % | | |

Scale up of the CEX Process: In the above results section, we present process modeling to demonstrate the feasibility and energy consumption of the crystallization and solvent recycling operations. However, the CEX results were performed in batch column mode. To address the scalability of the CEX operation we provide a discussion on scaling the system using the results obtained from the batch experiments. In the single column experiments, we demonstrated the conversion of KGA to GA via a cation exchange process with the DOWEX G26 resin. In this CEX step, H⁺ ions in the solid phase resin were displaced by K⁺ ions in the mobile phase, converting KGA to GA. The second pH increase in FIG. 3 indicates the KGA frontal curve. Typically, the KGA frontal curve is broad and overlaid with the GA curve due to the mass transfer resistance such as film mass transfer, axial dispersion, and intraparticle diffusion. Since the presence of KGA in the eluent prevents clean GA crystallization, it is critical to collect the GA eluent before the KGA frontal curve breaks through. Thus, for a large-scale continuous process, the design of the CEX process must consider the elution time of the mass transfer zone (MTZ) between GA and KGA. This can be modeled with the maximum loading volume (V_{f,max}) equation shown below.

$${V}_{f , 𝔪ax} = \frac{{q}_{e} {A}_{c}}{{C}_{{K}^{+}}} ( {L}_{c} - \frac{1}{2} {L}_{MTZ} ) \qquad ( 2 )$$

In Equation 2, C_(K\hat{ }+) is the concentration of K+ ion in a feed, V_MTZ is the volume of the MTZ, q_e is the resin capacity, V_c is the column volume, A_c is the cross-sectional area, L_MTZ is the length of MTZ.

Since the conversion of KGA to GA follows a displacement chromatography mechanism, L_MTZ in Equation 2 can be theoretically predicted from its analytical solution (Equation 3) assuming the column is sufficiently long and film mass transfer effect is negligible.

$${L}_{MTZ} = ( \frac{{E}_{b}}{{u}_{0}} + \frac{{ε}_{b} {u}_{0} {R}_{p}^{2}}{15 ( 1 - {ε}_{b} ) {ε}_{p} {D}_{P}} ) ( \frac{α + 1}{α - 1} ) ln \vert \frac{1 - θ}{θ} \vert \qquad ( 3 )$$

In equation 3, E_b is the axial dispersion coefficient, ε_b is the bed porosity, ε_p is the intraparticle porosity, R_p is the radius of resin particle, u_0 is the linear velocity, D_p is the intraparticle diffusion coefficient, a is the sorbent selectivity of K+ over H+, and θ is the cut off value of a breakthrough curve.

Equation 3 combined with Equation 2 represents the overall effect of system and operating parameters on the elution time for the length of MTZ (t_MTZ). For example, increasing the flowrate (=ε_b_u_0 A_c) or resins particle size (R_p) leads to an increased V_MTZ so that V_(f,max) will be reduced. Thus, for scaling up a CEX process, Equation 2 and 3 are useful to calculate the maximum loading volume when operating conditions are changed in large scale, but still run in a single column mode.

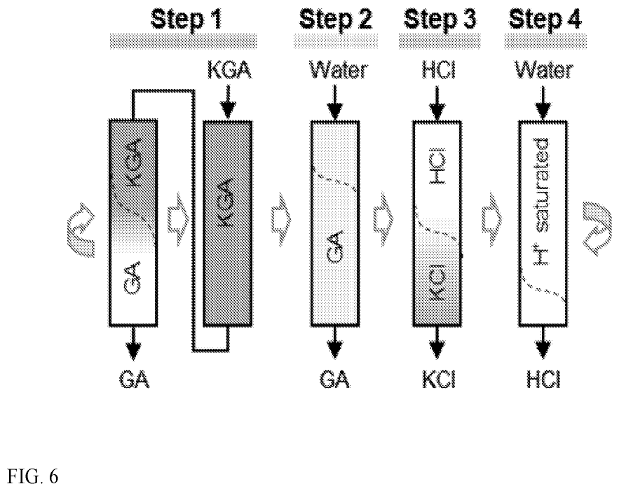

Alternatively, one can use multiple columns for a continuous process such as a carousel or periodic counter-current process, allowing full column utilization. In a continuous system, feed loading, washing, and regeneration steps occur at the same time, which increases the yield as well as the process productivity, generally by an order of magnitude.40 An example of a continuous CEX process for KGA conversion to GA is illustrated in FIG. 6. Since the feed loading time is critical in the process, Eq. (2) is useful to determine the column switching time in a continuous CEX process. From simulation studies, the value of D_p was estimated as 5.6×10⁻⁴ cm²/min. In order to determine the port switching time (t_sw) of the illustrated process in FIG. 6, a simple way is to set t_sw as the calculated maximum loading time for the given flowrate, feed concentration, and column size. Calculations that estimate a full scale continuous cation exchange process's parameters (including switch times and flowrates).

Comparison of this work to other glucarate isolation methods. In Table 5, the yields and purities of KGA and GA products from this study are compared with other representative methods reported in the literature. There are several different conversion methods to produce glucarate salts from glucose including; nitric acid oxidation, microorganisms (biocatalysis), catalytic oxidation, and electrochemical oxidation. Historically, nitric acid oxidation of glucose was the first approach developed to produce KGA but isolated solid recovery yields from this method are relatively low (41˜43%), Table 5. Another oxidation method is the use chlorine gas with a nitroxide catalyst (4-Acetamido-TEMPO) wherein, either pH adjustment or ethanol antisolvent precipitation methods were used to recover glucarate salts. Although it showed a high glucarate yield (70˜85%, Table 5), the products were contaminated with byproducts (e.g. chloride salts and tartaric acid) and the use of toxic chemicals and an expensive catalyst limits a large-scale process.

TABLE 5
Comparison of GA synthesis and separation methods in the literature.

| | Final | Separation | Recovery | Purity | |
|---|---|---|---|---|---|
| Synthesis Method | Product | Method | Yield [%] | [%] | Ref. |
| Fermentation | KGA₍ₛ₎ | pH adjustment with | >99.9 | 97.7 | This work |
| | | acetone antisolvent | | | |
| Glucose oxidation using | KGA₍ₛ₎ | pH adjustment | 41 | 99.2 | |
| nitric acid | | | | | |
| Glucose oxidation using | KGA₍ₛ₎ | pH adjustment | 43 | N.D. | |
| nitric acid | | | | | |
| Glucose nitroxide- | 1) KGA₍ₛ₎ | 1) pH adjustment | 1) 70 | 1) 90.9 | |
| mediated oxidation with | 2) NaGA₍ₛ₎ | 2) Ethanol antisolvent | 2) 85 | 2) 90 | |
| chlorine gas | | | | | |
| Glucose and myoinositol | GA₍ₛ₎ | IPA antisolvent | 71.1 | 98.3 | This work |
| | | crystallization with | | | |
| | | azeotropic drying | | | |
| Mock solution of KGA | GA₍ₛ₎ | Azeotropic drying | 98.7 | 99.9 | |

Biocatalysis methods using engineered microorganisms usually exhibit high glucose conversions (>99%) and selectivities to glucarate salts but suffer from lower yields (48%) and low titers (<10 g/L). Due to the high selectivity of biocatalysis methods the resulting broth is more amenable to achieving high recovery yield and purity in the downstream separation train. In this work the pH adjustment method with acetone addition resulted in a KGA recovery yield of >99.9% at a purity of >97.7% (Table 5). To our knowledge this is the highest reported recovery yield and purity of KGA from a reaction solution.

Catalytic oxidation and electrochemical oxidation methods have also been widely studied and achieved relatively higher conversions (>98%), but their reported yields vary (40-84%). Additionally, these methods were done at small scale less than 100 mL and the resulting glucarate concentration in the reaction solution ranges from 1.8 to 30.8 g/L, which is lower than that of our biological approach, and thus produces a more heterogeneous product solution. It should be noted that most of the approaches, except nitric acid or nitroxide oxidation, did not actually isolate products but reported the recovery yields and purities of glucarate salts or GA based on a final product concentration in solution and therefore could not be included in Table 5 for comparison. Furthermore, the catalytic oxidation methods were reported for the conversion of GA from glucose under high temperatures (80 ˜ 100° C.) and pressures (13.2 ˜ 40 bar). In these conditions, GA was readily lactonized, but the product speciation was unknown.

Due to the difficulty of isolating purified GA diacid crystals, recovery yields and purities of the diacid have been rarely reported. One notable approach that reported the isolation of purified GA is the work of Armstrong et. al. In that work, acetonitrile was used as an azeotropic distillation aid to recover GA from a KGA mock solution at recovery yields and purities of 98.7% and 99.9% respectively, Table 5. The methods, systems and compositions of matter disclosed herein are the first instance of isolated GA diacid crystals produced from real fermentation broth rather than from mock solutions. Our IPA antisolvent and azeotropic drying method recovered GA diacid crystals from fermentation broth at recovery yields of 71.1% and a purity of 98.3%. The recovery yield and purity is lower than that reported from Armstrong et. al. but could be the result of working with real fermentation derived material compared to mock solutions. Given the high recovery yields and purities of KGA and GA from our method using real fermentation broth, we hypothesize that our procedure can be broadly applied to solutions generated from other abiotic conversion technologies, but with potential additional considerations due to disparate impurities that are present in those solutions.

### CONCLUSION

Glucaric acid is regarded as a top-value added compound, however, the free acid form of GA is still not available in commercial markets due to difficulties in isolating the free acid. In certain embodiments disclosed herein, a downstream process was developed for producing and isolating pure KGA and GA crystals from fermentation broth. In the proposed process, antisolvent crystallization using acetone was applied to first recover KGA from the broth. In the KGA recovery step, adjusting pH to 3.5 by adding acid and acetone (30 vol %) as an antisolvent decreased the KGA solubility to almost zero, enabling selective precipitation of KGA and yielding high purity (95.6%) crystals. To isolate a pure crystalline GA, the solid KGA was first dissolved in water, acidified via a CEX process, and then the crystallization and isolation of GA were conducted using an IPA/water system. The added IPA at 87.5 wt. % acted as an azeotropic distillation aid and a concomitant antisolvent. Product characterization showed that acetone washing increased the purity of the GA product by removing lactone impurities, resulting in a GA recovery yield of 71% with 98.3% purity. To our knowledge, this is the largest quantity of isolated GA product (>2.2 g, FIG. 10) reported to date. Furthermore, the process modeling presented here provides a path towards industrial scale implementation. This modeling found the energy consumption was primarily from the solvent recycling distillation processes. A heat integration process will further reduce the energy footprint of the system. Lastly, the developed separations method and the reported physicochemical properties of GA could be useful to the separation of other highly oxygenated acids from both biotic and abiotic processes.

FIG. 7A shows the equilibration of glucaric acid (GA) and lactones with reaction coefficients. Each reaction coefficient was defined in Table 6. FIG. 7B was obtained by calculating the following algebraic equations simultaneously.

$$\frac{d {C}_{G A}}{d t} = - ( {k}_{2} + {k}_{3} ) {C}_{G A} + {k}_{1} {C}_{1 , 4 L} + {k}_{4} {C}_{6 , 3 L} \qquad ( S1 )$$

$$\frac{d {C}_{1 , 4 L}}{d t} = {k}_{2} {C}_{G A} - ( {k}_{1} + {k}_{8} ) {C}_{1 , 4 L} + {k}_{7} {C}_{D L} \qquad ( S2 )$$

$$\frac{d {C}_{6 , 3 L}}{d t} = {k}_{3} {C}_{G A} - ( {k}_{4} + {k}_{5} ) {C}_{6 , 3 L} + {k}_{6} {C}_{D L} \qquad ( S3 )$$

$$\frac{d {C}_{D L}}{dt} = {k}_{8} {C}_{1 , 4 L} - ( {k}_{6} + {k}_{7} ) {C}_{D L} + {k}_{5} {C}_{6 , 3 L} \qquad ( S4 )$$

Where 1,4 L denotes 1,4-lactone; 6,3 L denotes 6,3-lactone; and DL denotes 1,4:6,3-dilactone. The value of each rate constant was obtained from the literature and listed in Table 6.

TABLE 6
Rate constants for the equilibration of glucaric
acid and lactones at 300 K

| Rate constant | Reaction | Values (s⁻¹) |
|---|---|---|
| k₁ | 1,4-lactone → GA | 7.04 × 10⁻⁵ |
| k₂ | GA → 1,4-lactone | 4.84 × 10⁻⁵ |
| k₃ | GA → 6,3-lactone | 5.52 × 10⁻⁵ |
| k₄ | 6,3-lactone → GA | 8.70 × 10⁻⁵ |
| k₅ | 6,3-lactone → 1,4:6,3-dilactone | 1.74 × 10⁻⁴ |
| k₆ | 1,4:6,3-dilactone → 6,3-lactone | 1.97 × 10⁻³ |
| k₇ | 6,3-lactone → 1,4:6,3-dilactone | 6.64 × 10⁻⁴ |
| k₈ | 1,4:6,3-dilactone → 6,3-lactone | 5.01 × 10⁻⁵ |

In FIG. 12A the strong peaks at 1683 and 1725 cm⁻¹ in GA can be attributed to the two carboxylic acid C—O stretches. KGA contains these peaks as well, though with diminished intensity and the 1683 cm⁻¹ peak shifted to 1640 cm⁻¹ due to substitution of an acidic H+ with K+. Strong peaks at 3480, 3293, 3172 cm⁻¹ and the broad absorption between 3000-3500 cm⁻¹ correspond to the O—H groups of the backbone and acids, and the medium stretching peaks around 2950 cm⁻¹ can be attributed to the alkane C—H bonds.

Aspen Plus model for KGA and GA recovery system was built based on a UNIFAC model. Crystallization property setup was determined based on either literature or experimental data. Crystallization reactions for KGA and GA were set as KGA (I)→KGA(s) and GA (I)→GA(s), respectively. Solubility data was obtained from experimental results presented in FIG. 6. Solid molar volume (Vₘ) was calculated based on the unit cell parameters obtained from literature XRD data as shown below.

$${V}_{m} = \frac{{N}_{A}}{Z} {V}_{c e l l} = \frac{{N}_{A}}{2} abc \vert sin β \vert for monoclinic structure ( Z = 2 ) \qquad ( S5 )$$

Where N_A is Avogadro's number, Vcell is the unit cell volume, Z is the number of formula units per unit cell; a,b,c, and β are the unit cell dimensions. Eq. (S5) provides V_m for KGA and GA as 272.19 cm3/mol and 127.03 cm3/mol, respectively. Solid heat capacity (C_p) correlation parameters in Eq. S6 were obtained by fitting a C_p(T) curve measured from DSC curves in FIG. 17 and presented in Table 7.

$${C}_{p}^{s} = {C}_{1} + {C}_{2} T + {C}_{3} {T}^{2} + \frac{{C}_{4}}{T} + \frac{{C}_{5}}{{T}^{2}} + \frac{{C}_{6}}{\sqrt{T}} \qquad ( S6 )$$

TABLE 7
Input parameters for calculating Cp (T) in Aspen Plus

| | C₁ | C₂ | C₃ | C₄ | C₅ | C₆ |
|---|---|---|---|---|---|---|
| KGA | 0.1669 | 0.01668 | −4.3E−05 | 0 | 0 | 0 |
| GA | 0.2952 | 0.000385 | 2.94E−05 | 0 | 0 | 0 |

Please note that a solvent recovery step for GA, which is an extractive distillation process in FIG. 5B, was calculated using a NRTL model. For the extractive distillation, dimethylsulfoxide (DMSO) was used as an entrainer. The binary parameters for calculating activity coefficients were obtained from Arifin and Chien's data. Also, temperature and pressure of two flash distillations were determined based on the sensitivity analysis, which was set to separate the GA from solvent streams with >99.9% yield.

TABLE 8

| Binary parameters used in NRTL model | | | |
|---|---|---|---|
| comp, i | IPA | IPA | H2O |
| comp, j | H2O | DMSO | DMSO |
| aᵢᵢ | 0 | 0 | −1.2449 |
| aⱼᵢ | 0 | 0 | 1.7524 |
| bᵢ | 185.4 | 115.2787 | 586.801 |
| bⱼᵢ | 777.3 | −25.0123 | −1130.22 |
| Cᵢⱼ | 0.5 | 0.3 | 0.3 |

FIG. 19 shows the experimental data of KGA breakthrough curve and the simulation data. The simulation was conducted using Aspen Chromatography V10 simulator based on the experimental conditions. The simulated curve was well agreed with experimental data by fitting the intraparticle diffusion coefficient (Dp). The best fit of the curve provided the value of Dp as 5.6×10⁻⁴ cm²/min, which was presented with other simulation parameters in Table 9.

As an example, we design a CEX process at preparative scale with the column size of 10 cm inner diameter and 60 cm length and the same intrinsic parameters as presented in Table 9. Based on Equation 2 and 3, the maximum loading volume (V_(f,max)) of 0.1 M KGA for a single column is 75.6 L under the flowrate of 250 ml/min and 1% breakthrough cut. For designing a simple carousel process that switching all ports at the same time as illustrated in FIG. 7, we can use the calculated V_(f,max) and the flowrate for the loading step (step 1) such that the port switching time is set as 302 min, and the flowrate for other steps can be determined (usually faster than the loading flowrate) to elute all species out before the switching time.

TABLE 9
Simulation parameters used in Aspen Chromatography V10.
Note that default values are used for other parameters.

| | | | |
|---|---|---|---|
| Column length, Lc (cm) | 10 | Kinetic model | Particle MB |
| | | | (Rate model) |
| Column diameter (cm) | 1.6 | Film mass transfer | Wilson and |
| | | | Geankoplis |
| | | | correlation⁶ |
| Resin particle radius, Rₚ | 325 | Axial dispersion | Constant |
| (μm) | | | dispersion |
| Size exclusion factor | 1.0 | Axial dispersion | 1.0 × 10⁻³ |
| | | coefficient, Eₚ (cm²/min) | |
| Bed porosity, ε_{b} | 0.34 | Brownian diffusion | 5.6 × 10⁻³ |
| | | coefficient, D_{b} (cm²/min) | |
| Intraparticle porosity, εₚ | 0.27 | Intraparticle diffusion | 5.6 × 10⁻⁴ |
| | | coefficient, Dₚ (cm²/min) | |
| Isotherm model | Mass action equilibrium | PDE method | UDS1 |
| Sorbent selectivity, α | 2.54 | Axial elements | 100 nodes |
| Column capacity, qₑ | 2.0 | Collocation points in | 4 nodes |
| (mequiv./ml) | | particle phase | |
| Density (kg/m³) | 996 | Integration method | Implicit Euler |
| Viscosity (cP) | 0.89 | Tolerance (absolute) | 0.0005 |
| Feed concentration | 0.06 | Tolerance (relative) | 0.0005 |
| (mequiv./ml) | | | |
| Flowrate (ml/min) | 2.5 | Time step size | Variable (0.1 ~ |
| | | | 0.5) |

The foregoing disclosure has been set forth merely to illustrate the invention and is not intended to be limiting.

We claim:

1. A method for isolating monopotassium glucarate and glutaric acid from a fermentation broth, the method comprising contacting the fermentation broth with a first antisolvent and then isolating monopotassium glucarate by adjusting the pH of the fermentation broth and antisolvent mixture to about 3.5 through the addition of a sufficient amount of the antisolvent and an acid; and wherein the method further comprises dissolving the isolated monopotassium glucarate in water and acidifying the monopotassium glucarate and water solution using a cation exchange column and adding a second antisolvent wherein the acidified monopotassium glucarate and antisolvent solution is distilled and glutaric acid is isolated.

2. The method of claim 1 wherein the first antisolvent is acetone.

3. The method of claim 1 wherein the second antisolvent is isopropanol.

4. The method of claim 1 further comprising the step of isolating the second antisolvent after the distillation of the acidified monopotassium glucarate and antisolvent solution.

5. The method of claim 4 wherein the second antisolvent is isopropanol.

6. The method of claim 5 wherein the isopropanol recovery yield is greater than 99%.

7. The method of claim 1 wherein the addition of the second antisolvent to the acidified monopotassium glucarate solution creates an azeotropic solution.

8. The method of claim 1 further comprising the step of isolating the first antisolvent after recovering monopotassium glucarate.

9. The method of claim 8 wherein the first antisolvent is acetone.

10. The method of claim 9 wherein the acetone recovery yield is greater than 99%.

11. The method of claim 1 wherein the monopotassium glucarate is recovered with a yield of greater than 99.9%.

12. The method of claim 1 wherein the monopotassium glucarate is recovered with a purity of greater than 95%.

13. The method of claim 1 wherein the glucaric acid is recovered with a yield of greater than 71%.

14. The method of claim 1 wherein the glucaric acid is recovered with a purity of greater than 98%.

15. The method of claim 1 wherein the energy consumption of the method is less than about 20 MJ/kg for isolating monopotassium glucarate.

16. The method of claim 1 wherein the energy consumption of the method is less than about 1460 MJ/kg for isolating glucaric acid.

17. A method for isolating a carboxylic acid salt and a carboxylic acid from a fermentation broth, the method comprising contacting the fermentation broth with a first antisolvent and then isolating the carboxylic acid salt by adjusting the pH of the fermentation broth and antisolvent mixture to below 7 through the addition of a sufficient amount of the antisolvent and an acid; and wherein the method further comprises dissolving the isolated carboxylic acid salt in water and acidifying the carboxylic acid salt and water solution using a cation exchange column and adding a second antisolvent wherein the acidified carboxylic acid salt and antisolvent solution is distilled and the carboxylic acid is isolated.

18. The method of claim 17 wherein the addition of the second antisolvent to the acidified carboxylic acid salt solution creates an azeotropic solution.

19. The method of claim 17 wherein the carboxylic acid salt is recovered with a yield of greater than 99.9% and with a purity of greater than 95%.

20. The method of claim 17 wherein the glucaric acid is recovered with a yield of greater than 71% and with a purity of greater than 98%.
